(19) United States
(12) Patent Application Publication
(10) Pub. No.: US 2017/0283842 A1
(43) Pub. Date: Oct. 5, 2017

(54) METHODS OF PRODUCING BACTERIAL NANOCELLULOSE FROM CASSAVA BAGASSE

(71) Applicant: SHANGHAI ZHIYI INFORMATION TECHNOLOGY LTD, Shanghai (CN)

(72) Inventor: Feng Hong, Shanghai (CN)

(73) Assignee: SHANGHAI ZHIYI INFORMATION TECHNOLOGY LTD, Shanghai (CN)

(21) Appl. No.: 15/505,320

(22) PCT Filed: Aug. 29, 2014
(86) PCT No.: PCT/CN2014/085516

§ 371 Date: Feb. 21, 2017

(51) Int. Cl. C12P 19/04; C12R 1/02; C12R 1/025; C12R 1/865; C12R 1/065; C12R 1/38; C12R 1/41; C12R 1/645; C12R 1/01; C12R 1/05

(52) U.S. Cl. CPC C12P 19/04; C12R 1/01; C12R 1/02; C12R 1/025; C12R 1/05; C12R 1/065; C12R 1/38; C12R 1/41; C12R 1/645; C12R 1/865; C12R 1/00

(57) ABSTRACT

Methods, compositions, systems and kits relating to processing of cassava bagasse into bacterial feedstock, such as bacterial feedstock suitable for nanocellulose production, are disclosed. Cassava bagasse may be contacted with an acid catalyst or an enzymatic catalyst to produce a hydrolysate, which can be used to form a pre-fermentation medium. Incubation of the pre-fermentation medium with a first population of microorganisms yields a supernatant enriched in reducing sugars, which may be used to form a culture medium which can be used to support growth of a second population of microorganisms to form the nanocellulose.

### FIELD

Disclosed are methods and compositions for processing plant materials, such as cassava bagasse or cassava by-products, into feedstocks for nanocellulose production.

### BACKGROUND

Bacterial nanocellulose (BNC) is an extracellular biopolymer produced in a microbial fermentation process. Vinegar bacteria are commonly used in the production of BNC. BNC has many excellent properties, such as a high purity (free of lignin and hemicellulose), a high crystallinity, a high degree of polymerization, a nano-structured network, a high wet tensile strength, a high water-holding capacity, and good biocompatibility. These characteristics distinguish BNCs from plant cellulose. In view of these positive features, BNC is considered for applications in many different fields, such as biomedicine, food industry, cosmetics, advanced acoustic diaphragms, paper-making, and textile industry.

### SUMMARY

Disclosed herein are methods for producing nanocellulose, such as bacterial nanocellulose. In one embodiment, a method of producing nanocellulose includes contacting cassava bagasse with a catalyst to form a reaction mixture, wherein the catalyst is an acid catalyst, an enzymatic catalyst or a combination thereof; subjecting the reaction mixture to conditions sufficient to hydrolyze at least a portion of the cassava bagasse to produce cassava bagasse hydrolysate, preparing a pre-fermentation medium that includes the cassava bagasse hydrolysate; inoculating the pre-fermentation medium with a first population of microorganisms to produce an inoculated pre-fermentation medium; incubating the inoculated pre-fermentation medium to produce an enriched supernatant; collecting the enriched supernatant; preparing a culture medium that includes the enriched supernatant; inoculating the culture medium with a second population of microorganisms to produce an inoculated culture medium; and incubating the inoculated culture medium to produce nanocellulose.

In an additional embodiment, a method of producing bacterial nanocellulose includes treating at least one cassava by-product to form particles of cassava by-product having an average particle size of about 250 μm to about 420 μm in size; preparing a pre-fermentation medium that includes the particles of cassava by-product; inoculating the pre-fermentation medium with a first population of microorganism to produce an inoculated pre-fermentation medium; incubating the inoculated pre-fermentation medium to produce an enriched supernatant; collecting the enriched supernatant; preparing a culture medium that includes the enriched supernatant; inoculating the culture medium with a second population of microorganism to produce an inoculated culture medium; and incubating the inoculated culture medium to produce bacterial nanocellulose.

Additionally disclosed herein are methods of making culture media. In one embodiment, a method of making a culture medium includes contacting cassava bagasse with a catalyst to form a reaction mixture, wherein the catalyst is an acid catalyst, an enzymatic catalyst, or a combination thereof; subjecting the reaction mixture to conditions sufficient to hydrolyze at least a portion of the cassava bagasse to produce cassava bagasse hydrolysate; preparing a pre-fermentation medium including the cassava bagasse hydrolysate, inoculating the pre-fermentation medium with a population of microorganism to produce an inoculated pre-fermentation medium, incubating the inoculated pre-fermentation medium to produce an enriched supernatant, collecting the enriched supernatant, and preparing the culture medium with the enriched supernatant.

Additionally disclosed herein are culture media for the culturing of bacterial cells. In one embodiment, a culture medium in which bacterial cells can be cultured is provided, the culture medium including at least one nitrogen source and an enriched supernatant having reducing sugars at a concentration of about 5 g/L to about 300 g/L. Kits for the production of bacterial nanocellulose are also disclosed herein. In one embodiment, the kit includes a culture medium having at least one nitrogen source, an enriched supernatant having reducing sugars at a concentration of about 5 g/L to about 300 g/L, and a population of microorganisms selected from Gluconacetobacter xylinus, Gluconacetobacter hansenii, Gluconobacter oxydans, Rhizobium sp., Sarcina sp., Pseudomonas sp., Achromobacter sp., Alcaligenes sp., Aerobacter sp., Azotobacter sp., Agrobacterium sp., Seudomonas cepacia, Campylobacter jejuni, or a combination thereof.

Systems for production of bacterial nanocellulose are also disclosed herein. In one embodiment, a system for the production of bacterial nanocellulose includes a catalyst suitable for the hydrolysis of at least a portion of a sample of cassava bagasse, the catalyst being an acid catalyst, an enzymatic catalyst or both; a mixer configured to contact the catalyst with the cassava bagasse to produce cassava bagasse hydrolysate; a pre-fermentation container configured to contain an inoculated pre-fermentation medium that includes the cassava bagasse hydrolysate, a first nitrogen source, and a first population of microorganisms; an incubator configured to incubate the inoculated pre-fermentation medium in the pre-fermentation container to produce an enriched supernatant; and a culture medium container configured to contain an inoculated culture medium that includes the enriched supernatant, a second nitrogen source, and a second population of microorganisms, the incubator being further configured to incubate the inoculated culture medium in the culture medium container to produce bacterial nanocellulose.

The foregoing summary is illustrative only and is not intended to be in any way limiting. In addition to the illustrative aspects, embodiments, and features described above, further aspects, embodiments, and features will become apparent by reference to the drawings and the following detailed description.

### DETAILED DESCRIPTION

### General

Bacterial nanocellulose (BNC) is a product exhibiting desirable properties such as transparency, tensile strength, fiber-binding ability, biocompatibility, and biodegradability. Industrial production and application of BNC can be hindered by the high costs associated with culture media inoculated with microorganisms, and by relatively low yields from the production.

A variety of carbon sources can be used in BNC production, such as, monosaccharides (such as glucose and fructose), disaccharides (such as sucrose and maltose), and alcohols (such as ethanol, glycerol, and mannitol). These feedstocks can, however, be expensive, and may sometimes result in low yields of BNC, further elevating BNC production cost and limiting the scale of industrial manufacture of BNC.

Cassava (Manihot esculenta Cranz) is an important source of food and dietary calories for a large population in tropical countries throughout Asia, Africa and Latin America. World production of cassava has steadily increased from about 75 million tons in 1961-1965 to around 200 million tons more recently. Industrial processing of cassava is done mainly to isolate flour (which generates more solid residues) and starch (which generates more liquid residues) from the tubers. Thus, two types of wastes are generated from processing of cassava; solid waste and liquid waste. Solid wastes include peels and bagasse. Processing of 250-300 tons of cassava tubers yields about 1.6 tons of peels and about 280 tons of bagasse with a relatively high moisture content (85% by weight) and liquid wastes including wastewater (about 2655 m³) with about 1% solids by weight. Solid wastes (solid peels and cassava bagasse) are generally discarded in the environment as landfill without any treatment. Not only is their disposal an environmental concern, but, as disclosed herein, the discarded cassava could be used to facilitate low-cost BNC production.

Fibrous cassava bagasse residue contains about 30-50% weight/weight (w/w) starch or 40-65% (w/w) carbohydrates, 14-50% (w/w) fibers, 0.3-1.6% (w/w) proteins, 0.5-1% (w/w) lipids and 0.6-1.5% (w/w) ash on dry weight basis. Due to its rich organic nature and low ash content, it can serve as an ideal substrate for microbial fermentation for the production of BNC.

### Methods of Producing Bacterial Nanocellulose (BNC)

Several embodiments disclosed herein relate to methods of using residues generated in the cassava-processing industry (for example cassava bagasse and/or solid peels), to produce BNC or other related products, such as culture media for use in BNC production. Some embodiments of the present disclosure relate to methods of producing bacterial nanocellulose by contacting cassava bagasse with a catalyst, such as an acid catalyst, an enzymatic catalyst or a combination thereof, to form a reaction mixture. The reaction mixture can be subjected to conditions sufficient to allow the catalyst to hydrolyze at least a portion of the cassava bagasse to produce cassava bagasse hydrolysate. A pre-fermentation medium made up of, at least in part, the cassava bagasse hydrolysate, can be prepared and subsequently inoculated with a first population of microorganisms resulting in production of an inoculated pre-fermentation medium. The inoculated pre-fermentation medium may be incubated to produce an enriched supernatant, which can then be collected. A culture medium made up of, at least in part, the enriched supernatant, may be prepared and inoculated with a second population of microorganisms, resulting in production of an inoculated culture medium. The inoculated culture medium may be incubated to produce the bacterial nanocellulose.

In several embodiments, the method further includes separating any unhydrolyzed cassava bagasse, or a portion of the unhydrolyzed cassava bagasse, from the cassava bagasse hydrolysate before preparing the pre-fermentation medium. The separating may be performed by centrifuging, filtering, or both. In some embodiments, the method further includes adding the unhydrolyzed cassava bagasse into the reaction mixture before subjecting the reaction mixture to conditions sufficient to allow the catalyst to hydrolyze the cassava bagasse. During the subjecting step, at least a portion of the unhydrolyzed cassava bagasse that is added into the reaction mixture may be hydrolyzed to produce additional cassava bagasse hydrolysate.

In some embodiments, the method may also include fragmenting the cassava bagasse before contacting with the catalyst. In some embodiments, the cassava bagasse has an average particle size of about 250 μm to about 420 μm after the fragmenting. For example, the average particle size after the fragmenting may be about 250 μm, about 260 μm, about 270 μm, about 280 μm, about 290 μm, about 300 μm, about 310 μm, about 230 μm, about 330 μm, about 340 μm, about 350 μm, about 360 μm, about 370 μm, about 380 μm, about 390 μm, about 400 μm, about 410 μm, about 420 μm, or any size between these values. The fragmenting may be accomplished by cutting the cassava bagasse, grinding the cassava bagasse, or both.

In some embodiments, the method also includes reducing a water content of the cassava bagasse before the fragmenting. The water content can be reduced by crushing the cassava bagasse to release water from the cassava bagasse, and removing the water. The removal of the water can be achieved for example by filtration, centrifugation or other suitable methods. Additionally, in some embodiments, at least a portion of a cassava plant is crushed to form the cassava bagasse before reducing the water content of the cassava bagasse.

### Acid Hydrolysis and Detoxification

In several embodiments, the catalyst is an acid catalyst. The acid catalyst may be an inorganic acid, for example, inorganic acids such as H₂SO₄, HCl, H₃PO₄, HNO₃, or a combination thereof. The acid catalyst may also be an organic acid. For example, organic acids such as acetic acid, citric acid, phytic acid, heteropolyacid, or a combination thereof may be used. In some embodiments, the acid catalyst may be an aqueous acid solution having a concentration of about 0.2% to about 10% weight per volume (weight/volume). In some embodiments, the acid catalyst is an aqueous acid solution having a concentration of about 0.3% to about 7% weight/volume. For example, the aqueous acid solution may have a concentration of about 0.3% to about 0.5%, about 0.5% to about 0.7%, about 0.7% to about 1.0%, about 1.0% to about 2.0%, about 2.0% to about 3.0%, about 3.0% to about 4.0%, about 4.0% to about 5.0%, about 5.0% to about 6.0%, about 6.0% to about 7.0%, about 7.0% to about 8.0%, about 8.0% to about 9.0%, about 9.0% to about 10.0% weight/volume, or a concentration between any of the these ranges (including endpoints).

In several embodiments, the cassava bagasse and an aqueous form of the catalyst are present in the reaction mixture at a ratio of about 1:5 weight/volume to about 1:30 weight/volume, including about 1:5 weight/volume to about 1:10 weight/volume, about 1:10 weight/volume to about 1:15 weight/volume, about 1:15 weight/volume to about 1:20 weight/volume, about 1:20 weight/volume to about 1:25 weight/volume, about 1:25 weight/volume to about 1:30 weight/volume, or a ratio between any of the ranges ratios listed (including endpoints).

In some embodiments, contacting the cassava bagasse with the catalyst may include contacting for about 12 hours to about 24 hours, for example, about 12 hours to about 14 hours, about 14 hours to about 16 hours, about 16 hours to about 18 hours, about 18 hours to about 20 hours, about 20 hours to about 22 hours, about 22 hours to about 24 hours or any length of time between these ranges (including endpoints).

In some embodiments, where the catalyst is an acid catalyst, the subjecting step may include subjecting the reaction mixture to a temperature of about 25° C. to about 200° C. For example, the subject step may include subjecting the reaction mixture to a temperature of about 25° C. to about 40° C., about 40° C. to about 50° C., about 50° C. to about 70° C., about 70° C. to about 90° C., about 90° C. to about 110° C., about 110° C. about 130° C., about 130° C. to about 150° C., about 150° C. to about 170° C., about 170° C. to about 180° C., about 180° C. to about 190° C., about 190° C. to about 200° C., or a temperature between any of these ranges (including endpoints).

In some embodiments, where the catalyst is an acid catalyst, the subjecting step may occur for about 10 minutes to about 200 minutes. In some embodiments, the subjecting step may occur for about 30 minutes to about 180 minutes. In some embodiments, the subjecting step may occur for about 10 minutes to about 90 minutes. For example, in some embodiments, the subjecting step may occur for about 10 minutes to about 15 minutes, about 15 minutes to about 20 minutes, about 20 minutes to about 25 minutes, about 25 minutes to about 30 minutes, about 30 minutes to about 35 minutes, about 35 minutes to about 40 minutes, about 40 minutes to about 45 minutes, about 45 minutes to about 50 minutes, about 50 minutes to about 55 minutes, about 55 minutes to about 60 minutes, about 60 minutes to about 65 minutes, about 65 minutes to about 70 minutes, about 70 minutes to about 75 minutes, about 75 minutes to about 80 minutes, about 80 minutes to about 85 minutes, about 85 minutes to about 90 minutes, about 90 minutes to about 105 minutes, about 105 minutes to about 120 minutes, about 120 minutes to about 135 minutes, about 135 minutes to about 150 minutes, about 150 minutes to about 165 minutes, about 165 minutes to about 180 minutes, about 180 minutes to about 200 minutes, or a time period between any of these ranges (including endpoints).

According to certain embodiments, the length of time and temperature of the hydrolysis reaction is inversely correlated. For example, in those embodiments in which a higher temperature is used, the length of time required for the progression of the hydrolysis reaction may be reduced. Determination of the precise times and temperatures can be readily made without undue experimentation.

In some embodiments, where the catalyst is an acid catalyst, the method of producing bacterial nanocellulose may further include detoxifying the cassava bagasse hydrolysate before preparing the pre-fermentation medium. In some embodiments, the detoxifying step may include adjusting a pH value of the cassava bagasse hydrolysate to a first acidic pH with an acid, contacting the cassava bagasse hydrolysate with activated carbon; and separating the activated carbon from the cassava bagasse hydrolysate. In several embodiments, the acid used to adjust the pH of the cassava bagasse hydrolysate to the first acidic pH during the detoxification step is H₂SO₄, HCl, HNO₃, H₃PO₄, acetic acid, citric acid or a combination thereof. In some embodiments, the first acidic pH of the cassava bagasse hydrolysate is less than about pH 6. In some embodiments, the first acidic pH may be about pH 5. For example, the first acidic pH may be about pH 4.5, about pH 4.6, about pH 4.7, about pH 4.8, about pH 4.9, about pH 5, about pH 5.1, about pH 5.2, about pH 5.3, about pH 5.4, about pH 5.5, about pH 5.6, about pH 5.7, about pH 5.8, about pH 5.9, about pH 6.0, or a pH value between any of these values.

In some embodiments, contacting the cassava bagasse hydrolysate with the activated carbon in the detoxifying step may include mixing the activated carbon with the cassava bagasse hydrolysate for about 2 minutes to about 15 minutes. For example, the activated carbon is mixed with the hydrolysate for about 2 minutes to about 4 minutes, about 4 minutes to about 5 minutes, about 5 minutes to about 6 minutes, about 6 minutes to about 7 minutes, about 7 minutes about 9 minutes, about 9 minutes about 11 minutes, about 11 minutes to about 13 minutes, about 13 minutes to about 15 minutes, or a time period between any of these ranges (including endpoints). In several embodiments, contacting the cassava bagasse hydrolysate with the activated carbon in the detoxifying step may include mixing the activated carbon with the cassava bagasse hydrolysate for about 5 minutes.

In some embodiments, the activated carbon may be present in the cassava bagasse hydrolysate at a concentration of about 1% weight/volume to about 20% weight/volume. For example, the activated carbon may be present in the cassava bagasse hydrolysate at a concentration of about 1% weight/volume to about 3% weight/volume, of about 3% weight/volume to about 6% weight/volume, of about 6% weight/volume to about 8% weight/volume, of about 8% weight/volume to about 10% weight/volume, about 10% weight/volume to about 15% weight/volume, of about of about 15% weight/volume to about 20% weight/volume, or a concentration between any of these ranges (including endpoints).

In several embodiments, the activated carbon is present in the cassava bagasse hydrolysate at a concentration of about 2% weight/volume to about 10% weight/volume.

In some embodiments, separating the activated carbon from the hydrolysate is accomplished using filtration, centrifugation, or both.

In some embodiments, after separating the activated carbon from the cassava bagasse hydrolysate, the pH value of the cassava bagasse hydrolysate is adjusted to a second acidic pH with an acid. In several embodiments, the acid used to adjust the pH value of the cassava bagasse hydrolysate to the second acidic pH is H₂SO₄, HCl, HNO₃, H₃PO₄, acetic acid, citric acid or a combination thereof In some embodiments, the second acidic pH of the cassava bagasse hydrolysate is about pH 5. For example, the second acidic pH may be about pH 4.5, about pH 4.6, about pH 4.7, about pH 4.8, about pH 4.9, about pH 5, about pH 5.1, about pH 5.2, about pH 5.3, about pH 5.4, about pH 5.5, or a pH value between any of these values. In some embodiments, the first acidic pH and the second acidic pH are different. In some embodiments, the first acidic pH and the second acidic pH are substantially the same.

In some embodiments, where the catalyst is an acid catalyst, the method of producing bacterial nanocellulose may further include detoxifying the cassava bagasse hydrolysate with an enzyme before preparing the pre-fermentation medium. In some embodiments, the detoxifying step may include adjusting a pH value of the cassava bagasse hydrolysate to a first pH and contacting the cassava bagasse hydrolysate with an oxidase enzyme. The oxidase enzyme can, for example, be laccase. The enzyme can, in some embodiments, be a peroxidase with hydrogen peroxide. In several embodiments the method may further include adjusting the pH to a second pH after contacting the cassava bagasse hydrolysate with the oxidase enzyme. In some embodiments, the first pH and the second pH may be the same or may be different.

In several embodiments, the pH of the cassava bagasse hydrolysate is adjusted to the first pH using a base. For example the pH may be adjusted using sodium hydroxide (NaOH), calcium hydroxide Ca(OH)₂, ammonia, or a combination thereof In some embodiments, the first pH is less than or equal to about pH 6. In some embodiments, the first pH may be about pH 5. For example, the first pH may be about 4.5, about 4.6, about 4.7, about 4.8, about 4.9, about 5, about 5.1, about 5.2, about 5.3, about 5.4, about 5.5, about 5.6, about 5.7, about 5.8, about 5.9, about 6.0 or a pH value between any of these values.

In some embodiments, the oxidase enzyme may have an enzyme unit of about 1U to about 100U. For example, the enzymatic catalyst may have an enzyme unit of about 1U to about 2U, about 2U to about 2.25U, about 2.25U to about 2.5U, about 2.5U to about 2.75U, about 2.75U to about 3U, about 3U to about 5U, about 5U to about 10U, about 10U to about 20U, about 20U to about 30U, about 30U to about 40U, about 40U to about 50U, about 50U to about 60U, about 60U to about 70U, about 70U to about 80U, about 80U to about 90U, about 90U to about 100U, or an enzyme unit between any of these ranges (including endpoints).

In some embodiments, the oxidase enzyme may be added to the cassava bagasse hydrolysate in an amount of about 1% to about 20% (by volume), including about 1% to about 2%, about 2% to about 4%, about 4% to about 6%, about 6% to about 8%, about 8% to about 10%, about 10% to about 12%, about 12% to about 14%, about 14% to about 16%, about 16% to about 18%, about 18% to about 20%, or a concentration between any of these ranges (including endpoints).

In some embodiments, contacting the cassava bagasse hydrolysate with the oxidase enzyme involves incubating the cassava bagasse hydrolysate and the oxidase enzyme for about 30 minutes to about 48 hours. For example, the incubation may be for about 30 minutes to about 1 hour, about 1 hour to about 4 hours, about 4 hours to about 8 hours, about 8 hours to about 12 hours, about 12 hours to about 16 hours, about 16 hours to about 20 hours, about 20 hours to about 24 hours, about 24 hours to about 28 hours, about 28 hours to about 32 hours, about 32 hours to about 36 hours, about 36 hours to about 40 hours, about 40 hours to about 44 hours, about 44 hours to about 48 hours, or for a time period between any of these ranges (including endpoints).

In some embodiments, incubating the cassava bagasse hydrolysate with the oxidase enzyme may include subjecting the cassava bagasse hydrolysate and the oxidase enzyme to a temperature of about 25° C. to about 90° C. For example, the temperature can be about 25° C. to about 30° C., about 30° C. to about 35° C., about 35° C. to about 40° C., about 40° C. to about 45° C., about 45° C. to about 50° C., about 50° C. to about 55° C., about 55° C. to about 60° C., about 60° C. to about 65° C., about 65° C. to about 70° C., about 70° C. to about 75° C., about 75° C. to about 80° C., about 80° C. to about 85° C., about 85° C. to about 90° C. or a temperature between any of these ranges (including endpoints).

In several embodiments, after contacting the cassava bagasse hydrolysate with the oxidase enzyme, the pH of the mixture is adjusted to a second pH using an acid (or a base if needed). Suitable acids for adjusting the pH include H₂SO₄, HCl, HNO₃, H₃PO₄, acetic acid, citric acid, or a combination thereof. In some embodiments, the second pH of the cassava bagasse hydrolysate is less than about pH 6. In some embodiments the second pH is equal to the first pH. In some embodiments, the second pH is about pH 5. For example, the second pH may be about pH 4.5, about pH 4.6, about pH 4.7, about pH 4.8, about pH 4.9, about pH 5, about pH 5.1, about pH 5.2, about pH 5.3, about pH 5.4, about pH 5.5, about pH 5.6, about pH 5.7, about pH 5.8, about pH 5.9, about pH 6.0 or a pH value between any of these values.

### Enzymatic Hydrolysis

In some embodiments, the catalyst may be an enzymatic catalyst. In some embodiments, the enzymatic catalyst may be a saccharification enzyme. For example, the saccharification enzyme can be a cellulase, hemicellulase, xylanase, endoglucanase, cellobiase, amylase, glucan glucohydrolase, glucoamylase, protease, pectinase, lipase or a combination thereof.

In some embodiments, the enzymatic catalyst may have an enzyme unit of about 1U to about 700U. For example, the enzymatic catalyst may have an enzyme unit of about 1U to about 50U, about 50U to about 100U, about 100U to about 150U, about 150U to about 200U, about 200U to about 250U, about 250U to about 300U, about 300U to about 350U, about 400U to about 450U, about 450U to about 500U, about 500U to about 550U, about 550U to about 600U, about 600U to about 650U, about 650U to about 700U, or an enzyme unit between any of these ranges (including endpoints).

In some embodiments, where the catalyst is an enzymatic catalyst, the subjecting step may occur for about 30 minutes to about 48 hours. For example, the subjecting step may be for about 30 minutes to about 1 hour, about 1 hour to about 4 hours, about 4 hours to about 8 hours, about 8 hours to about 12 hours, about 12 hours to about 16 hours, about 16 hours to about 20 hours, about 20 hours to about 24 hours, about 24 hours to about 28 hours, about 28 hours to about 32 hours, about 32 hours to about 36 hours, about 36 hours to about 40 hours, about 40 hours to about 44 hours, about 44 hours to about 48 hours, or a time period between any of these ranges (including endpoints).

In some embodiments, the subjecting step may include subjecting the reaction mixture to a temperature of about 25° C. to about 90° C. For example, the reaction mixture may be subjected to temperatures of about 25° C. to about 30° C., about 30° C. to about 35° C., about 35° C. to about 40° C., about 40° C. to about 45° C., about 45° C. to about 50° C., about 50° C. to about 55° C., about 55° C. to about 60° C., about 60° C. to about 65° C., about 65° C. to about 70° C., about 70° C. to about 75° C., about 75° C. to about 80° C., about 80° C. to about 85° C., about 85° C. to about 90° C. or a temperature between any of these ranges (including endpoints). According to certain embodiments, the length of time and temperature of the hydrolysis reaction is inversely correlated. For example, in those embodiments in which a higher temperature is used, the length of time required for the progression of the hydrolysis reaction may be reduced. Similarly, alterations in the amount of enzyme used can result in changes in the time and/or the temperature required (for example, increased amounts of enzymes can achieve hydrolysis in reduced amounts of time). Determination of the precise times and temperatures can be readily made without undue experimentation.

In several embodiments, the cassava bagasse and the enzymatic catalyst are present in the reaction mixture at a ratio of about 1:5 weight/volume to about 1:30 weight/volume, including about 1:5 weight/volume to about 1:10 weight/volume, about 1:10 weight/volume to about 1:15 weight/volume, about 1:15 weight/volume to about 1:20 weight/volume, about 1:20 weight/volume to about 1:25 weight/volume, about 1:25 weight/volume to about 1:30 weight/volume, or a ratio between any of these ranges (including endpoints).

### Fragmentation of Cassava By-product

Instead of producing cassava bagasse hydrolysates, fragmented cassava by-products can alternatively be used to prepare the pre-fermentation medium. Also provided herein are methods for producing bacterial nanocellulose using pre-fermentation medium prepared from fragmented cassava by-products. The method may include treating at least one cassava by-product to form particles of cassava by-product having an average particle size of about 250 μm to about 420 μm, preparing a pre-fermentation medium including the particles of cassava by-product, inoculating the pre-fermentation medium with a first population of microorganisms, incubating the inoculated pre-fermentation medium to produce an enriched supernatant, collecting the enriched supernatant, preparing a culture medium including the enriched supernatant, inoculating the culture medium with a second population of microorganisms to produce an inoculated culture medium; and incubating the inoculated culture medium to produce bacterial nanocellulose. Advantageously, such methods do not require generation of an acidic or enzymatic hydrolysate. In some embodiments, the at least one cassava by-product includes cassava bagasse, cassava peels, water derived from cassava processing, starch containing cassava processing effluents, or a combination thereof.

In some embodiments, the average particle size of the cassava by-product may be about 250 μm, 260 μm, 270 μm, 280 μm, 290 μm, 300 μm, 310 μm, 230 μm, 330 μm, 340 μm, 350 μm, 360 μm, 370 μm, 380 μm, 390 μm, 400 μm, 410 μm, 420 μm, or a size between any of these values. The fragmenting may be accomplished by cutting the cassava by-product, grinding the cassava by-product, or both.

In some embodiments, the method also includes reducing a water content of the cassava by-product before the fragmenting. In some such embodiments, the water content is reduced by crushing the cassava by-product to release water from the cassava by-product, and removing the water. The removal of the water can be achieved for example by filtration, centrifugation or other suitable methods. Additionally, in some embodiments, at least a portion of a cassava plant is crushed to form the at least one cassava by-product before reducing the water content.

### Preparation of Pre-fermentation Medium and Enriched Supernatant

In some embodiments, the preparation of the pre-fermentation medium involves the addition of at least one nitrogen source to the cassava bagasse hydrolysate. In several embodiments, the at least one nitrogen source is present in the pre-fermentation medium in an amount of about 0.1% weight/volume to about 3% weight/volume. For example, depending on the embodiment, the at least one nitrogen source may be present in the pre-fermentation medium in an amount of about 0.1% weight/volume to about 0.3% weight/volume, about 0.3% weight/volume to about 0.5% weight/volume, about 0.5% weight/volume to about 0.7% weight/volume, about 0.7% weight/volume to about 0.9% weight/volume, about 0.9% weight/volume to about 1.1% weight/volume, about 1.1% weight/volume to about 1.3% weight/volume, about 1.3% weight/volume to about 1.5% weight/volume, about 1.5% weight/volume to about 1.7% weight/volume, about 1.7% weight/volume to about 1.9% weight/volume, about 1.9% weight/volume to about 2.1% weight/volume, about 2.1% weight/volume to about 2.3% weight/volume, about 2.3% weight/volume to about 2.5% weight/volume, about 2.5% weight/volume to about 2.7% weight/volume, about 2.7% weight/volume to about 2.9% weight/volume, about 2.9% weight/volume to about 3% weight/volume, or a percentage between any of these ranges (including endpoints).

In several embodiments, the preparation of the pre-fermentation medium includes adding at least one nitrogen source to the particles of cassava by-product. For example, depending on the embodiment, the at least one nitrogen source may be added to the cassava by-product particles to reach an amount of nitrogen of about 0.1% weight/volume to about 3% weight/volume, for example, about 0.1% weight/volume to about 0.3% weight/volume, about 0.3% weight/volume to about 0.5% weight/volume, about 0.5% weight/volume to about 0.7% weight/volume, about 0.7% weight/volume to about 0.9% weight/volume, about 0.9% weight/volume to about 1.1% weight/volume, about 1.1% weight/volume to about 1.3% weight/volume, about 1.3% weight/volume to about 1.5% weight/volume, about 1.5% weight/volume to about 1.7% weight/volume, about 1.7% weight/volume to about 1.9% weight/volume, about 1.9% weight/volume to about 2.1% weight/volume, about 2.1% weight/volume to about 2.3% weight/volume, about 2.3% weight/volume to about 2.5% weight/volume, about 2.5% weight/volume to about 2.7% weight/volume, about 2.7% weight/volume to about 2.9% weight/volume, about 2.9% weight/volume to about 3% weight/volume, or a percentage between any of these ranges (including endpoints).

In several embodiments, the at least one nitrogen source may be an organic nitrogen. In several embodiments, the organic nitrogen is peptone, yeast extract, tryptone, or a combination thereof. In some embodiments, the pre-fermentation medium includes peptone in an amount of about 0.1% to about 0.5% (including about 0.1% to about 0.3%, about 0.3% to about 0.5%, or a percentage between any of these ranges, including endpoints), yeast extract in an amount of about 0.3% to about 0.7% (including about 0.3% to about 0.5%, about 0.5% to about 0.7% or a percentage between any of these ranges, including endpoints), and glucose in an amount of about 1.0% to about 5% (including about 1.0% to about 2.0%, about 2.0% to about 3.0%, about 3.0% to about 4.0%, about 4.0% to about 5.0% or a percentage between any of these ranges, including endpoints), each based on weight/volume.

In one embodiment, the pre-fermentation medium includes peptone in an amount of about 0.3%, yeast extract in an amount of about 0.5%, and glucose in an amount of about 2.5%, weight/volume.

After forming the pre-fermentation medium, a first population of microorganisms can be added to the pre-fermentation medium to produce an inoculated pre-fermentation medium. In some embodiments, the first population of microorganisms is present in the pre-fermentation medium at a concentration of about 5% by volume to about 15% by volume. For example, the concentration of the first population of microorganisms can be about 5% to about 7% by volume, about 7% to about 9% by volume, about 9% to about 11% by volume, about 11% to about 13% by volume, about 13% to about 15% by volume, or a concentration between any of these ranges (including endpoints). Depending on the embodiment, the first population of microorganisms may be a mold or a fungus. In several embodiments, the first population of microorganisms may be Rhizopus sp., Aspergillus niger, or both.

In some embodiments, the inoculated pre-fermentation medium may be incubated according to any of the incubation times, temperatures or methods disclosed in more detail below. In some embodiments, the inoculated pre-fermentation medium may be incubated at a temperature of about 20° C. to about 40° C., including about 37° C. For example, the inoculated pre-fermentation medium may be incubated at a temperature of about 20° C. to about 25° C. , about 25° C. to about 30° C.; about 30° C. to about 35° C., about 35° C. to about 37° C., about 37° C. to about 40° C., or a temperature between any of these ranges (including endpoints). In some embodiments, the inoculated pre-fermentation medium may be incubated for about 3 days to about 20 days. For example, the inoculated pre-fermentation medium may be incubated for about 3 days to about 5 days, about 5 days to about 7 days, about 7 days to about 10 days, about 10 days to about 15 days, about 15 days to about 20 days, or a time period between any of these ranges (including endpoints). The incubation can be performed in a static incubator, or an incubator capable of moving the pre-fermentation medium.

Collection of the enriched supernatant that results from the incubation of the inoculated pre-fermentation medium may be performed by a variety of methods. For example, the enriched supernatant can be collected by filtration, centrifugation or both. In some embodiments, both filtration and centrifugation are used.

In some embodiments, the enriched supernatant may have a reducing sugar concentration of about 5 g/L to about 300 g/L. For example, the reducing sugar concentration can be about 5 g/L to about 25 g/L, about 25 g/L to about 50 g/L, about 50 g/L to about 75 g/L, about 75 g/L to about 100 g/L, about 100 g/L to about 125 g/L, about 125 g/L to about 150 g/L, about 150 g/L to about 175 g/L, about 175 g/L to about 200 g/L, about 200 g/L to about 225 g/L, about 225 g/L to about 250 g/L, about 250 g/L to about 275 g/L, about 275 g/L to about 300 g/L, or a concentration between any of these ranges (including endpoints).

### Culture Media Production and Generation of Bacterial Nanocellulose

In several embodiments, the preparation of the culture medium includes adding at least one nitrogen source, at least one trace element, or both, to the enriched supernatant. In some embodiments, the nitrogen source may be present in the culture medium in an amount of about 0.1% weight/volume to about 3% weight/volume. For example, depending on the embodiment, the at least one nitrogen source may be present in the culture medium in an amount of about 0.1% weight/volume to about 0.3% weight/volume, about 0.3% weight/volume to about 0.5% weight/volume, about 0.5% weight/volume to about 0.7% weight/volume, about 0.7% weight/volume to about 0.9% weight/volume, about 0.9% weight/volume to about 1.1% weight/volume, about 1.1% weight/volume to about 1.3% weight/volume, about 1.3% weight/volume to about 1.5% weight/volume, about 1.5% weight/volume to about 1.7% weight/volume, about 1.7% weight/volume to about 1.9% weight/volume, about 1.9% weight/volume to about 2.1% weight/volume, about 2.1% weight/volume to about 2.3% weight/volume, about 2.3% weight/volume to about 2.5% weight/volume, about 2.5% weight/volume to about 2.7% weight/volume, about 2.7% weight/volume to about 2.9% weight/volume, about 2.9% weight/volume to about 3% weight/volume, or a percentage between any of these ranges (including endpoints).

In several embodiments, the at least one nitrogen source may be an organic nitrogen. In several embodiments, the organic nitrogen is peptone, yeast extract, tryptone, or a combination thereof In some embodiments, the culture medium includes peptone in an amount of about 0.1% weight/volume to about 0.5% weight/volume (including about 0.1% weight/volume to about 0.3% weight/volume, about 0.3% weight/volume to about 0.5% weight/volume, or a concentration between any of these ranges, including endpoints), yeast extract in an amount of about 0.3% weight/volume to about 0.7% weight/volume (including 0.3% weight/volume to about 0.5% weight/volume, about 0.5% weight/volume to about 0.7% weight/volume or a concentration between any of these ranges, including endpoints), and glucose in an amount of about 1.0% weight/volume to about 5% weight/volume (including about 1.0% weight/volume to about 2.0% weight/volume, about 2.0% weight/volume to about 3.0% weight/volume, about 3.0% weight/volume to about 4.0% weight/volume, about 4.0% weight/volume to about 5.0% weight/volume, or a concentration between any of these ranges, including endpoints). In some embodiments, if the glucose levels are below about 1.0% weight/volume (for example, because the first population of microorganisms over consumed the glucose), the culture medium can be supplemented with additional glucose to achieve a glucose concentration of about 1% weight/volume to about 5% weight/volume. In one embodiment, the culture medium includes peptone in an amount of about 0.3% weight/volume, yeast extract in an amount of about 0.5%, weight/volume and glucose in an amount of about 2.5%, weight/volume.

After forming the culture medium, a second population of microorganisms can be added to the culture medium to produce an inoculated culture medium. In some embodiments, the second population of microorganisms is present in the culture medium at a concentration of about 5% by volume to about 15% by volume. For example, in some embodiments the concentration of the second population of microorganisms can be about 5% by volume to about 7% by volume, about 7% by volume to about 9% by volume, about 9% by volume to about 11% by volume, about 11% by volume to about 13% by volume, about 13% by volume to about 15% by volume, or a concentration between any of these ranges (including endpoints). Depending on the embodiment, the second population of microorganisms may be one or more types of microorganisms. In some embodiments, the second population of microorganisms may be Gluconacetobacter xylinus, Gluconacetobacter hansenii, Gluconobacter oxydans, Rhizobium sp., Sarcina sp., Pseudomonas sp., Achromobacter sp., Alcaligenes sp., Aerobacter sp., Azotobacter sp., Agrobacterium sp., Seudomonas cepacia, Campylobacter jejuni, or a combination thereof.

In some embodiments, the inoculated culture medium may be incubated according to any of the incubation times, temperatures or methods disclosed in more detail below. For example, in some embodiments, the inoculated culture medium may be incubated at a temperature of about 20° C. to about 40° C., including about 37° C. For example, in some embodiments, the inoculated culture medium may be incubated at a temperature of about 20° C. to about 25° C. , about 25° C. to about 30° C.; about 30° C. to about 35° C., about 35° C. to about 37° C., about 37° C. to about 40° C., or a temperature between any of these ranges (including endpoints). In some embodiments, the inoculated culture medium may be incubated for about 3 days to about 20 days. For example, in some embodiments, the inoculated culture medium may be incubated for about 3 days to about 5 days, about 5 days to about 7 days, about 7 days to about 10 days, about 10 days to about 15 days, about 15 days to about 20 days, or a time period between any of these ranges (including endpoints). The incubation can be performed in a static incubator or a movable incubator.

In several embodiments, the bacterial nanocellulose that is produced is harvested from the culture medium. In some embodiments, the harvested bacterial nanocellulose is contacted with a base under conditions that allow removal of residual first population of microorganisms, residual second population of microorganisms and residual culture medium. In some such embodiments, the base is contacted with the bacterial nanocellulose and may be heated to a temperature of about 70° C. to about 120° C. For example, the base may be contacted with the bacterial nanocellulose at a temperature of about 70° C. to about 75° C., about 75° C. to about 80° C., about 80° C. to about 85° C., about 85° C. to about 90° C., about 90° C. to about 95° C., about 95° C. to about 100° C., about 100° C. to about 105° C., about 105° C. to about 110° C., about 110° C. to about 115° C., about 115° C. to about 120° C., or a temperature between any of these ranges (including endpoints).

In several embodiments, the base is contacted with the bacterial nanocellulose for about 90 minutes to about 150 minutes. For example, the base may be contacted with the bacterial nanocellulose for about 90 minutes to about 100 minutes, about 100 minutes to about 110 minutes, about 110 minutes to about 120 minutes, about 120 minutes to about 130 minutes, about 130 minutes to about 140 minutes, about 140 minutes to about 150 minutes, or a time period between any of these ranges (including endpoints).

In several embodiments, the base with which the bacterial nanocellulose is contacted may be NaOH, KOH, NH₄OH, or a combination thereof In some embodiment, the base is in aqueous form having a concentration of about 0.5% to about 8% by weight. For example, in some embodiments the aqueous form of the base has a concentration of about 0.5% to about 1.0%, about 1.0% to about 1.5%, about 1.5% to about 2.0%, about 2.0% to about 2.5%, about 2.5% to about 3.0%, about 3.0% to about 3.5%, about 3.5% to about 4.0%, about 4.0% to about 4.5%, about 4.5% to about 5.0%, about 5.0% to about 5.5%, about 5.5% to about 6.0%, about 6.0% to about 6.5%, about 6.5% to about 7.0%, about 7.0% to about 7.5%, about 7.5% to about 8%, (each by weight), or a concentration between any of these ranges (including endpoints). In several embodiments, rather than using a base, deionized water may be used.

### Media Sterilization

The pre-fermentation medium may be sterilized before inoculating with the first population of microorganisms. Likewise, the culture medium may be sterilized before inoculating with the second population of microorganisms.

In some embodiments, the sterilization of the pre-fermentation medium is accomplished by autoclaving the pre-fermentation medium at a temperature of about 100° C. to about 130° C. for a time of about 10 minutes to about 60 minutes. In some embodiments, where the pre-fermentation is prepared from particles of cassava by-product, the sterilization of the pre-fermentation medium is accomplished by autoclaving the pre-fermentation medium at a temperature of about 100° C. to about 130° C. for a time of about 10 minutes to about 60 minutes. In some embodiments, the sterilization of the culture medium is accomplished by autoclaving the culture medium at a temperature of about 100° C. to about 130° C. for a time of about 10 minutes to about 60 minutes.

In several embodiments, sterilization by autoclaving may occur at a temperature of about 100° C. to about 115° C., about 115° C. to about 120° C., about 120° C. to about 125° C. , about 125° C. to about 130° C. or a temperature between any of these ranges (including endpoints). In several embodiments, the sterilization by autoclaving may occur for a time period of about 10 minutes to about 60 minutes, including about 10 minutes to about 20 minutes, about 20 minutes to about 30 minutes, about 30 minutes to about 40 minutes, about 40 minutes to about 50 minutes, about 50 minutes to about 60 minutes, or a time period between any of these ranges (including endpoints).

In additional embodiments, filtration can be used (either in place of, or in conjunction with autoclaving) to sterilize the pre-fermentation medium and/or culture medium. In some embodiments, the sterilizing includes filtering the culture medium or pre-fermentation medium with sterile filters. For example, the pre-fermentation medium and/or culture medium can be sterilized by passage through a filter with a pore size of about 0.2 μm to about 0.45 μm, including about 0.2 μm to about 0.25 μm, about 0.25 μm to about 0.3 μm, about 0.3 μm to about 0.35 μm, about 0.35 μm to about 0.4 μm, about 0.4 μm to about 0.45 μm, or a pore size between any of these ranges (including endpoints).

### Incubation Conditions

The incubation conditions described herein can be applied equally to either the inoculated pre-fermentation medium or the inoculated culture medium. Time periods, temperatures, incubator types and the like are interchangeable and applicable to incubation of either media without undue experimentation.

In some embodiments, the inoculated pre-fermentation medium or the inoculated culture medium may be incubated at a temperature of about 20° C. to about 40° C. For example, the inoculated medium may be incubated at temperature of about 20° C. to about 22° C., about 22° C. to about 24° C., about 24° C. to about 26° C., about 26° C. to about 28° C., about 28° C. to about 30° C., about 30° C. to about 32° C., about 32° C. to about 34° C., about 34° C. to about 36° C., about 36° C. to about 38° C., about 38° C. to about 40° C., or a temperature between any of these ranges (including endpoints). In one embodiment, the inoculated medium may be incubated at temperature of about 37° C.

In several embodiments, the inoculated medium is incubated for about 3 days to about 20 days. For example, the incubating can be performed for about 3 to about 5 days, about 5 days to about 7 days, about 7 days to about 10 days, about 10 days to about 14 days, about 14 days to about 18 days, about 18 days to about 20 days, or a time period between any of these ranges (including endpoints).

In some embodiments, inoculated medium is incubated in a static incubator. In some embodiments, inoculated medium is incubated in an incubator capable of moving the medium, such as a shaking incubator, a rotating incubator, an oscillating incubator or a rocking incubator. In one embodiment, the rotating incubator has a rotation speed of about 100 rpm to about 500 rpm. For example, the rotation speed of the rotating incubator may be from about 100 rpm to about 120 rpm, about 120 rpm to about 140 rpm, about 140 rpm to about 160 rpm, about 160 rpm to about 180 rpm, about 180 rpm to about 200 rpm, about 200 rpm to about 220 rpm, about 220 rpm to about 240 rpm, about 240 rpm to about 250 rpm, about 250 rpm to about 275 rpm, about 275 rpm to about 300 rpm, about 300 rpm to about 350 rpm, about 350 rpm to about 400 rpm, about 400 rpm to about 450 rpm, about 450 rpm to about 500 rpm, or a rotation speed between any of these ranges (including endpoints). In one embodiment the inoculated medium is incubated in a rotating incubator with a rotation speed of about 160 rpm to about 250 rpm.

### Culture Medium Compositions

Some embodiments relate to culture media in which bacterial cells are cultured. In some embodiments, a culture medium for culturing bacterial cells may include at least one nitrogen source, and an enriched supernatant containing reducing sugars. As discussed above, the enriched supernatant may be produced from the inoculating the pre-fermentation medium with a population of microorganisms, and incubating the inoculated pre-fermentation medium. The pre-fermentation may be prepared from a cassava bagasse hydrolysate, produced from either acid hydrolysis or enzymatic hydrolysis. The pre-fermentation may alternatively be prepared from particles of cassava by-products. In several embodiments, the pre-fermentation may be detoxified before inoculating with the population of microorganisms. For example, where the cassava bagasse hydrolysate is produced from acid hydrolysis, activated carbon particles may be used to detoxify the hydrolysate. In several embodiments, the microorganisms used to generate the enriched supernatant may be a population of Rhizopus sp., Aspergillus niger, or both.

In several embodiments, the reducing sugars are present in the enriched supernatant at a concentration of about 5 g/L to about 300 g/L, including the concentrations disclosed in more detail above.

In several embodiments, the nitrogen source added to the enriched supernatant to result in a nitrogen concentration of about 0.1% to about 3%, weight/volume in the culture medium, including the nitrogen concentrations disclosed in more detail above.

In several embodiments, the at least one nitrogen source may be an organic nitrogen. In several embodiments, the organic nitrogen is peptone, yeast extract, tryptone, or a combination thereof. In some embodiments, the culture medium further includes at least one trace element. In some embodiments the at least one trace element is calcium, phosphorus, manganese, magnesium, or combinations of these trace elements. In some embodiments, the culture medium includes glucose in an amount of about 1% to about 5% weight/volume. In some embodiments, the at least one nitrogen source includes peptone and yeast extract, and the peptone is present in the culture medium in an amount of about 0.1% to about 0.5% weight/volume, and the yeast extract is present in the culture medium in an amount of about 0.3% to about 0.7% weight/volume.

### Kits for Bacterial Nanocellulose Production

Some embodiments relate to kits for producing bacterial nanocellulose. In some embodiments, the kit includes a culture medium including at least one nitrogen source, and an enriched supernatant containing reducing sugars at a concentration of about 5 g/L to about 300 g/L, including the concentrations disclosed in more detail above. The kit may further include a population of microorganisms selected from Gluconacetobacter xylinus, Gluconacetobacter hansenii, Gluconobacter oxydans, Rhizobium sp., Sarcina sp., Pseudomonas sp., Achromobacter sp., Alcaligenes sp., Aerobacter sp., Azotobacter sp., Agrobacterium sp., Seudomonas cepacia, Campylobacter jejuni, or a combination thereof. In some embodiments, the kit further includes at least one trace element. The kit may contain instructions for inoculating the culture medium with the population of microorganisms to produce an inoculated culture medium, and for incubating the inoculated culture medium to produce the bacterial nanocellulose. The culture medium, nitrogen source, enriched supernatant, microorganisms and other components that form the kit, may be in accordance with those as described above.

In several embodiments, the enriched supernatant is a result of the incubation of one or more of Rhizopus sp. and Aspergillus niger in the pre-fermentation medium.

In several embodiments, the nitrogen source is present in the in the culture medium at a concentration of about 0.1% weight/volume to about 3% weight/volume.

In several embodiments, the at least one nitrogen source may be an organic nitrogen. In several embodiments, the organic nitrogen is peptone, yeast extract, tryptone, or a combination thereof.

### Systems for Bacterial Nanocellulose Production

Systems for the production of bacterial nanocellulose are provided. The system for bacterial nanocellulose production may include a catalyst suitable for hydrolysis of at least a portion of a sample of cassava bagasse to produce cassava bagasse hydrolysate, wherein the catalyst is an acid catalyst, an enzymatic catalyst or a combination thereof; a mixer configured to contact the catalyst with the cassava bagasse to produce cassava bagasse hydrolysate; a pre-fermentation container configured to contain an inoculated pre-fermentation medium that includes the cassava bagasse hydrolysate, at least one nitrogen source, and a first population of microorganisms; an incubator configured to incubate the inoculated pre-fermentation medium in the pre-fermentation container to produce an enriched supernatant; and a culture medium container configured to contain an inoculated culture medium that includes the enriched supernatant, at least one nitrogen source, and a second population of microorganisms, the incubator being further configured to incubate the inoculated culture medium in the culture medium container to produce bacterial nanocellulose.

The mixer can be any mixer that can contact the catalyst with the cassava bagasse and may for example be a container with a stirrer, a container mounted onto a shaker, or any other suitable mixers. The pre-fermentation container and the culture medium container can be any container capable of containing the respective medium, and can for example be a petri-dish of various sizes depending on scale of bacterial nanocellulose production. The catalyst, nitrogen source, first population of microorganisms, second population of microorganisms, incubator, pre-fermentation medium, culture medium and other components described for the system may be in accordance with those as described above.

### Bacterial Nanocellulose Testing

The tensile strength of the bacterial nanocellulose may be measured. In some embodiments, the bacterial nanocellulose is harvested from the culture medium before measuring the tensile strength. In some embodiments, before tensile strength measurement, the bacterial nanocellulose may be soaked in a basic solution such as, for example, a 0.5% to 2% NaOH solution, weight/volume. For example, the NaOH solution may have a concentration of about 0.5% to about 0.75%, about 0.75% to about 1.0%, about 1.0% to about 1.25%, about 1.25% to about 1.5%, about 1.5% to about 1.75%, about 1.75% to about 2.0%, or a concentration, based on weight/volume, between any of these ranges (including endpoints).

The bacterial nanocellulose may also be heated at temperatures between about 60° C. and 100° C., for a time period of about 30 minutes to about 240 minutes. For example, the bacterial nanocellulose may be heated to about 60° C., about 70° C., about 80° C., about 90° C., about 100° C., or any temperature between any of these temperatures. The duration of heating may, in some embodiments, be about 30 minutes to about 60 minutes, about 60 minutes to about 90 minutes, about 90 minutes to about 120 minutes, about 120 minutes to about 150 minutes, about 150 minutes to about 180 minutes, about 180 minutes to about 240 minutes, or a duration between any of these ranges (including endpoints). The soaking of the bacterial nanocellulose in the basic solution and the heating can remove impurities such as residual culture medium and microorganisms. In some embodiments, the bacterial nanocellulose may be subdivided into smaller portions for analysis of tensile strength. In some embodiments the tensile strength of wet bacterial nanocellulose may be measured directly, such as by using a universal testing machine (H5K-S, Hounsfield Test Equipment Ltd, UK) operating at suitable parameters established in the art. The tensile strength (in megapascal, MPa, or N/mm²) may be calculated, for example, by dividing the tensile force by the area of the cross section of the bacterial nanocellulose tested or using other calculations known to one of skill in the art. The tensile strength of the bacterial nanocellulose, in some embodiments, is about 0.01 MPa to about 0.1 MPa, for example, from about 0.01 MPa to about 0.02 MPa, about 0.02 MPa to about 0.03 MPa, about 0.03 MPa to about 0.04 MPa, about 0.04 MPa to about 0.05 MPa, about 0.05 MPa to about 0.06 MPa, about 0.06 MPa to about 0.07 MPa, about 0.07 MPa to about 0.08 MPa, about 0.08 MPa to about 0.09 MPa, about 0.09 MPa to about 0.1 MPa, or a tensile strength between any of these ranges (including endpoints). The bacterial nanocellulose, in some embodiments, may be dried prior to testing tensile strength (or subjected to further processing). In several embodiments, the dry weight of bacterial nanocellulose obtained using the methods and compositions disclosed herein is about 5 g/L to about 20 g/L, for example, about 5 g/L to about 7 g/, about 7 g/L to about 9 g/L, about 9 g/L to about 11 g/L, about 11 g/L to about 13 g/L, about 13 g/L to about 15 g/L, about 15 g/L to about 17 g/L, about 17 g/L to about 18 g/L, about 18 g/L to about 20 g/L, or a dry weight between any of these ranges (including endpoints). Depending on the scale of production, larger amounts of bacterial nanocellulose may be produced.

### EXAMPLES

### Example 1

### Hydrolysis with Inorganic Acid

Cassava bagasse is mixed with 4% weight/volume diluted sulfuric acid aqueous solution for 12 hours in a container. The ratio of solid (cassava bagasse) to liquid (sulfuric acid aqueous solution) is adjusted to 1:15 weight/volume. The hydrolysis reaction is then allowed to proceed at 130° C. for 140 minutes.

### Example 2

### High Temperature Hydrolysis with Inorganic Acid

Cassava bagasse is mixed with 4% weight/volume diluted sulfuric acid aqueous solution for 12 hours in a container. The ratio of solid (cassava bagasse) to liquid (sulfuric acid aqueous solution) is adjusted to 1:15 weight/volume. The hydrolysis reaction is then allowed to proceed at 200° C. for 30 minutes.

### Example 3

### Low Temperature Hydrolysis with Inorganic Acid

Cassava bagasse is mixed with 4% weight/volume diluted sulfuric acid aqueous solution for 12 hours in a container. The ratio of solid (cassava bagasse) to liquid (sulfuric acid aqueous solution) is adjusted to 1:15 weight/volume. The hydrolysis reaction is then allowed to proceed at 80° C. for 180 minutes.

### Example 4

### Hydrolysis with Organic Acid

Cassava bagasse is mixed with 4% w/v diluted acid acetic acid aqueous solution for 12 hours in a container. The ratio of solid (cassava bagasse) to liquid (acetic acid aqueous solution) is adjusted to 1:15 weight/volume. The hydrolysis reaction is then allowed to proceed at 130° C. for about 140 minutes.

### Example 5

### High Temperature Hydrolysis with Organic Acid

Cassava bagasse mixed with 4% w/v diluted acid acetic acid aqueous solution for 12 hours in a container. The ratio of solid (cassava bagasse) to liquid (acetic acid aqueous solution) is adjusted to 1:15 weight/volume. The hydrolysis reaction is then allowed to proceed at 200° C. for 30 minutes.

### Example 6

### Low Temperature Hydrolysis with Organic Acid

Cassava bagasse is mixed with 4% w/v diluted acid acetic acid aqueous solution for 12 hours in a container. The ratio of solid (cassava bagasse) to liquid (acetic acid aqueous solution) is adjusted to 1:15 weight/volume. The hydrolysis reaction is then allowed to proceed at 80° C. for 180 minutes.

### Example 7

### Enzymatic Hydrolysis With a Saccharification Enzyme

Cassava bagasse is mixed with 300U of cellulase in a container. The ratio of solid (cassava bagasse) to liquid (cellulase) is adjusted to 1:20 weight/volume. Reactions are allowed to proceed at 50° C. for 48 hours.

### Example 8

### Enzymatic Hydrolysis With an Elevated Concentration of a Single Saccharification Enzyme

Cassava bagasse is mixed with 700U of cellulase in a container. The ratio of solid (cassava bagasse) to liquid (cellulase) is adjusted to 1:20 weight/volume. Reactions are allowed to proceed at 50° C. for 24 hours.

### Example 9

### Low Temperature Enzymatic Hydrolysis With a Single Saccharification Enzyme

Cassava bagasse is mixed with 300U of endoglucanase in a container. The ratio of solid (cassava bagasse) to liquid (endoglucanase) is adjusted to 1:20 weight/volume. Reactions are allowed to proceed at 45° C. for 72 hours.

### Example 10

### Elevated Temperature Enzymatic Hydrolysis With a Single Saccharification Enzyme

Cassava bagasse is mixed with 300U of endoglucanase in a container. The ratio of solid (cassava bagasse) to liquid (endoglucanase) is adjusted to 1:20 weight/volume. Reactions are allowed to proceed at 55° C. for about 24 hours.

### Example 11

### Enzymatic Hydrolysis Using Mixtures of Saccharification Enzymes

Cassava bagasse is mixed with a total of 500U of a mixture of saccharification enzymes including cellulase, hemicellulose, xylanase, protease, endoglucanase, cellobiase, lipase, amylase, glucan glucohydrolase, and glucoamylase in a container. The ratio of solid (cassava bagasse) to liquid (saccharification enzymes) is 1:20 weight/volume. Reactions are allowed to proceed at 50° C. for 48 hours.

### Example 12

### Enzymatic Hydrolysis Using Mixtures of Saccharification Enzymes at an Elevated Concentration

Cassava bagasse is mixed with a total of 700U of a mixture of saccharification enzymes including cellulase, hemicellulose, xylanase, protease, endoglucanase, cellobiase, lipase, amylase, glucan glucohydrolase, and glucoamylase in a container. The ratio of solid (cassava bagasse) to liquid (saccharification enzymes) is 1:20 weight/volume. Reactions are allowed to proceed at 50° C. for 24 hours.

### Example 13

### Low Temperature Enzymatic Hydrolysis Using Mixtures of Saccharification Enzymes

Cassava bagasse is mixed with a total of 500U of a mixture of saccharification enzymes including cellulase, hemicellulose, xylanase, protease, endoglucanase, cellobiase, lipase, amylase, glucan glucohydrolase, and glucoamylase in a container. The ratio of solid (cassava bagasse) to liquid (saccharification enzymes) is 1:20 weight/volume. Reactions are allowed to proceed at 45° C. for 72 hours.

### Example 14

### Elevated Temperature Enzymatic Hydrolysis Using Mixtures of Saccharification Enzymes

Cassava bagasse is mixed with a total of 500U of a mixture of saccharification enzymes including cellulase, hemicellulose, xylanase, protease, endoglucanase, cellobiase, lipase, amylase, glucan glucohydrolase, and glucoamylase in a container. The ratio of solid (cassava bagasse) to liquid (saccharification enzymes) is 1:20 weight/volume. Reactions are allowed to proceed at 55° C. for 24 hours.

### Example 15

### Detoxification Treatment Using Sodium Hydroxide and Laccase

The pH value of cassava bagasse acid hydrolysate from any one of Examples 1 to 6, is adjusted to pH 5.0 by addition of NaOH. Thereafter, laccase (2.75 U/mL) is added to the pH adjusted hydrolysate to a 10% (by volume) concentration. That mixture is incubated at 30° C. for 12 hours, and then the pH is adjusted to 5.0 by using H₂SO₄.

### Example 16

### Detoxification Treatment Using Calcium Hydroxide and Laccase

The pH value of cassava bagasse acid hydrolysate from any one of Examples 1 to 6 is adjusted to pH 5.0 by addition of Ca(OH)₂. Thereafter, laccase (2.75 U/mL) is added to the pH adjusted hydrolysate to a 10% (by volume) concentration. That mixture is incubated at 30° C. for 12 hours, and then the pH is adjusted to 5.0 by using H₂SO₄.

### Example 17

### Detoxification Treatment Using Ammonia and Laccase

The pH value of cassava bagasse acid hydrolysate from any one of Examples 1 to 6 is adjusted to pH 5.0 by addition of ammonia. Thereafter, laccase (2.75 U/mL) is added to the pH adjusted hydrolysate to a 10% (by volume) concentration. That mixture is incubated at 30° C. for 12 hours, and then the pH is adjusted to 5.0 by using H₂SO₄.

### Example 18

### Detoxification Treatment Using H₂SO₄ and Activated Carbon

The pH value of cassava bagasse acid hydrolysate from any one of Examples 1 to 6, is adjusted to pH 5.0 by addition of H₂SO₄, and mixing the pH-adjusted hydrolysate with activated carbon for 5 minutes. The activated carbon is added to the hydrolysate in a 5% (w/v) ratio. The activated carbon is removed from the hydrolysate and the pH is adjusted to 5.0 by addition of H₂SO₄.

### Example 19

### Detoxification Treatment Using HCl and Activated Carbon

The pH value of cassava bagasse acid hydrolysate from any one of Examples 1 to 6, is adjusted to pH 5.0 by addition of HCl, and mixing the pH-adjusted hydrolysate with activated carbon for 5 minutes. The activated carbon is added to the hydrolysate in a 5% (w/v) ratio. The activated carbon is removed from the hydrolysate and the pH is adjusted to 5.0 by addition of HCl.

### Example 20

### Detoxification Treatment Using HNO₃ and Activated Carbon

The pH value of cassava bagasse acid hydrolysate from any one of Examples 1 to 6, is adjusted to pH 5.0 by addition of HNO₃, and mixing the pH-adjusted hydrolysate with activated carbon for 5 minutes. The activated carbon is added to the hydrolysate in a 5% (w/v) ratio. The activated carbon is removed from the hydrolysate and the pH is adjusted to 5.0 by addition of HNO₃.

### Example 21

### Detoxification Treatment Using H₃PO₄ and Activated Carbon

The pH value of cassava bagasse acid hydrolysate from any one of Examples 1 to 6, is adjusted to pH 5.0 by addition of H₃PO₄, and mixing the pH-adjusted hydrolysate with activated carbon for 5 minutes. The activated carbon is added to the hydrolysate in a 5% (w/v) ratio. The activated carbon is removed from the hydrolysate and the pH is adjusted to 5.0 by addition of H₃PO₄.

### Example 22

### Detoxification Treatment Using Acetic Acid and Activated Carbon

The pH value of cassava bagasse acid hydrolysate from any one of Examples 1 to 6, is adjusted to pH 5.0 by addition of acetic acid, and mixing the pH-adjusted hydrolysate with activated carbon for 5 minutes. The activated carbon is added to the hydrolysate in a 5% (w/v) ratio. The activated carbon is removed from the hydrolysate and the pH is adjusted to 5.0 by addition of acetic acid.

### Example 23

### Detoxification Treatment Using Citric Acid and Activated Carbon

The pH value of cassava bagasse acid hydrolysate from any one of Examples 1 to 6, is adjusted to pH 5.0 by addition of citric acid, and mixing the pH-adjusted hydrolysate with activated carbon for 5 minutes. The activated carbon is added to the hydrolysate in a 5% (w/v) ratio. The activated carbon is removed from the hydrolysate and the pH is adjusted to 5.0 by addition of citric acid.

### Example 24

### Detoxification Treatment Using H₂SO₄, Citric Action and Activated Carbon

The pH value of cassava bagasse acid hydrolysate from any one of Examples 1 to 6, is adjusted to pH 5.0 by addition of H₂SO₄, and mixing the pH-adjusted hydrolysate with activated carbon for 5 minutes. The activated carbon is added to the hydrolysate in a 5% (w/v) ratio. The activated carbon is removed from the hydrolysate and the pH is adjusted to 5.0 by addition of citric acid.

### Example 25

### Direct Production of Pre-Fermentation Medium Using Fragmented Cassava Bagasse

Cassava bagasse and/or cassava peels are fragmented to form particles having an average size of 330 μm. The fragmented particles are used directly as a pre-fermentation medium for production of an enriched supernatant.

### Example 26

### Production of Culture Medium of Cassava Bagasse

Detoxified acid hydrolysate of cassava bagasse from any one of Examples 15 to 24 (which forms a pre-fermentation medium), enzyme hydrolysate of cassava bagasse from any one of examples 7 to 14 (which forms a pre-fermentation medium), or the pre-fermentation medium from Example 25, is combined with (weight/volume) 0.3% peptone, 0.5% yeast extract, and 2.5% glucose. The resultant mixture is sterilized with a sterile filter and inoculated with Rhizopus sp. and/or Aspergillus niger and incubated to generate a supernatant enriched with reducing sugars. The enriched supernatant is supplemented with (weight/volume) 0.3% peptone and 0.5% yeast extract and sterilized with a sterile filter to form a culture medium for culturing nanocellulose producing bacteria.

### Example 27

### Bacterial Nanocellulose Production

Nanocellulose producing bacteria (one or more of Gluconacetobacter xylinus, Gluconacetobacter hansenii, Gluconobacter oxydans, Rhizobium sp., Sarcina sp., Pseudomonas sp., Achromobacter sp., Alcaligenes sp., Aerobacter sp., Azotobacter sp., Agrobacterium sp., Seudomonas cepacia, and Campylobacter jejuni) are inoculated into the sterilized culture medium from Example 26 until the inoculum is present at 10% (by volume). The inoculated culture medium is incubated at 37° C. in an oscillating incubator (set at about 200 rpm) for 10 days. Bacterial nanocellulose is harvested by filtration. Thereafter, the bacterial nanocellulose is washed with deionized water and the dry weight of the nanocellulose is determined after drying at 105° C.±0.5° C. for 24 h. The resultant dry weight of the bacterial nanocellulose is about 8 g/L to about 18 g/L.

### Example 28

### Bacterial Nanocellulose Production

Nanocellulose producing bacteria (one or more of Gluconacetobacter xylinus, Gluconacetobacter hansenii, Gluconobacter oxydans, Rhizobium sp., Sarcina sp., Pseudomonas sp., Achromobacter sp., Alcaligenes sp., Aerobacter sp., Azotobacter sp., Agrobacterium sp., Seudomonas cepacia, and Campylobacter jejuni) are inoculated into the sterilized culture medium from Example 26 until the inoculum is present at 10% (by volume). The inoculated culture medium is incubated at 37° C. in an oscillating incubator (set at about 200 rpm) for 10 days. Bacterial nanocellulose is harvested by filtration. Thereafter, the bacterial nanocellulose is washed with a base and the dry weight of the nanocellulose is determined after drying at 105° C. ±0.5° C. for 24 h. The resultant dry weight of the bacterial nanocellulose is about 8 g/L to about 18 g/L.

### Example 29

### Tensile Strength Measurement

Nanocellulose producing bacteria are inoculated into aliquots of sterilized culture media from Examples 26 until the inoculum is present at 5% (by volume). Each inoculated culture medium is incubated at 30° C. in a static incubator for about 10 days. Bacterial nanocellulose was harvested. The bacterial nanocellulose is washed with deionized water to remove impurities (for example, culture medium and trapped bacterial cells) and the dry weight of the nanocellulose is determined after drying at 105° C.±0.5° C. for 24 h. The resultant dry weight of the bacterial nanocellulose averages about 8 g/L to about 18 g/L.

Before tensile strength measurement, the bacterial nanocellulose is soaked in 1% NaOH solution and heated at 80° C. for 120 minutes to further remove any residual impurities (for example, culture medium and trapped bacterial cells). The bacterial nanocellulose is cut into 40 mm long and 10 mm wide strips for analysis of tensile strength. The tensile strength of wet BNC is measured by using a universal testing machine (H5K-S, Hounsfield Test Equipment Ltd, UK) operating at a crosshead speed of 50 mm/min. All data for determination of tensile strength are collected under the same conditions. The tensile strength (in megapascal, MPa, or N/mm²) is calculated by dividing the tensile force by the area of the cross section of the BNC strip. Each test is performed by using 10 samples and mean values of the strength of BNC are given. The strength of BNC averages 0.03-0.06 Mpa.

The Examples above demonstrate that bacterial nanocellulose can be produced from biomass materials, such as waste cassava bagasse, which would otherwise be discarded. The cassava bagasse provides a low cost feedstock for producing bacterial nanocellulose, and the resulting product exhibited good tensile strength and yield.

In view of the disclosure and Examples discussed above, it shall be appreciated that bacterial nanocellulose produced by the methods disclosed herein possesses many desirable properties. For example, bacterial nanocellulose produced by the disclosed methods exhibits a desirable degree of transparency, thereby allowing its incorporation into other compositions without significant detriment or alteration of the coloring of those compositions. The bacterial nanocellulose produced by the disclosed methods exhibits desirable tensile strength, allowing its use in contexts requiring a material to resist forces that might damage or compromise other nanocellulose compositions. Additionally, bacterial nanocellulose produced by the disclosed methods exhibits excellent adaptability to living bodies, and biodegradability. This presents advantageous qualities for use in tissue engineering applications (for example, implants). Moreover, the bacterial nanocellulose produced by the disclosed methods is produced from what would otherwise be a significant source of environmental waste, thereby reducing adverse ecological impacts and reducing the cost of bacterial nanocellulose production.

With respect to the use of substantially any plural and/or singular terms herein, those having skill in the art can translate from the plural to the singular and/or from the singular to volume of wastewater can be received in the plural as is appropriate to the context and/or application. The various singular/plural permutations may be expressly set forth herein for sake of clarity.

It will be understood by those within the art that, in general, terms used herein, and especially in the appended claims (for example., bodies of the appended claims) are generally intended as “open” terms (for example., the term “including” should be interpreted as “including but not limited to,” the term “having” should be interpreted as “having at least,” the term “includes” should be interpreted as “includes but is not limited to,” and so on). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (for example., the bare recitation of “two recitations,” without other modifiers, means at least two recitations, or two or more recitations). Furthermore, in those instances where a convention analogous to “at least one of A, B, and C, and so on” is used, in general such a construction is intended in the sense one having skill in the art would understand the convention (for example., “a system having at least one of A, B, and C” would include but not be limited to systems that have A alone, B alone, C alone, A and B together, A and C together, B and C together, and/or A, B, and C together, and so on). In those instances where a convention analogous to “at least one of A, B, or C, and so on” is used, in general such a construction is intended in the sense one having skill in the art would understand the convention (for example., “a system having at least one of A, B, or C” would include but not be limited to systems that have A alone, B alone, C alone, A and B together, A and C together, B and C together, and/or A, B, and C together, and so on). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof Any listed range can be easily recognized as sufficiently describing and enabling the same range being broken down into at least equal halves, thirds, quarters, fifths, tenths, and so on. As a non-limiting example, each range discussed herein can be readily broken down into a lower third, middle third and upper third, and so on. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

1. A method of producing bacterial nanocellulose, the method comprising:
contacting cassava bagasse with a catalyst to form a reaction mixture, wherein the catalyst is an acid catalyst, an enzymatic catalyst or a combination thereof to produce cassava bagasse hydrolysate;
preparing a pre-fermentation medium comprising the cassava bagasse hydrolysate;
inoculating the pre-fermentation medium with a first population of microorganisms to produce an inoculated pre-fermentation medium;
incubating the inoculated pre-fermentation medium to produce an enriched supernatant;
collecting the enriched supernatant;
preparing a culture medium comprising the enriched supernatant;
inoculating the culture medium with a second population of microorganisms to produce an inoculated culture medium; and
incubating the inoculated culture medium to produce bacterial nanocellulose.

2. The method of claim 1, further comprising separating any unhydrolyzed cassava bagasse from the cassava bagasse hydrolysate before preparing the pre-fermentation medium.

3.-4. (canceled)

5. The method of claim 1, further comprising fragmenting the cassava bagasse before contacting with the catalyst, wherein the fragmenting comprises cutting the cassava bagasse, grinding the cassava bagasse, or both and wherein the cassava bagasse has an average particle size of about 250 μm to about 420 μm after the fragmenting.

6.-9. (canceled)

10. The method of claim 1, wherein the catalyst is an acid catalyst.

11. (canceled)

12. The method of claim 10, wherein the acid catalyst is an aqueous acid solution having a concentration of about 0.2% to about 10% weight/volume.

13. (canceled)

14. The method of claim 12, wherein the cassava bagasse and an aqueous form of the acid catalyst are present in the reaction mixture at a ratio of about 1:5 to about 1:30 weight/volume.

15. The method of claim 1, wherein contacting the cassava bagasse with the acid catalyst comprises contacting the cassava bagasse with the acid catalyst for about 12 hours to about 24 hours.

16. The method of claim 1, wherein the contacting step further comprises subjecting the reaction mixture comprising cassava bagasse and the acid catalyst to a temperature of about 25° C. to about 200° C. for about 10 minutes to about 200 minutes.

17. The method of claim 1, further comprising detoxifying the cassava bagasse hydrolysate before preparing the pre-fermentation medium, wherein detoxifying the cassava bagasse hydrolysate comprises:
adjusting the pH value of the cassava bagasse hydrolysate to a first acidic pH with an acid;
contacting the cassava bagasse hydrolysate with activated carbon; and
separating the activated carbon from the cassava bagasse hydrolysate.

18.-19. (canceled)

20. The method of claim 17, wherein the first acidic pH is less than about pH 6.

21. (canceled)

22. The method of claim 17, wherein contacting the cassava bagasse hydrolysate with activated carbon comprises mixing the activated carbon with the cassava bagasse hydrolysate for about 2 minutes to about 15 minutes, and wherein the activated carbon is present in the cassava bagasse hydrolysate in an amount of about 1% weight/volume to about 20% weight/volume.

23.-26. (canceled)

27. The method of claim 17, further comprising adjusting the pH of the cassava bagasse hydrolysate to a second acidic pH after separating the activated carbon from the hydrolysate, wherein the second acidic pH is about pH 5.

28. (canceled)

29. The method of claim 1, wherein the enzymatic catalyst is a saccharification enzyme selected from cellulase, hemicellulase, xylanase, endoglucanase, cellobiase, amylase, glucan glucohydrolase, glucoamylase, protease, pectinase, lipase and a combination thereof.

30.-31. (canceled)

32. The method of claim 1, wherein the cassava bagasse and the enzymatic catalyst are present in the reaction mixture at a ratio of about 1:5 to about 1:30 weight/volume and wherein the enzymatic catalyst has an enzyme unit of about 1U to about 700U.

33. The method of claim 1, wherein the contacting step further comprises subjecting the reaction mixture comprising cassava bagasse and the enzymatic catalyst to a temperature of about 25° C. to about 90° C. for about 30 minutes to about 48 hours.

34.-38. (canceled)

39. The method of claim 1, wherein the pre-fermentation medium comprises peptone in an amount of about 0.1% to about 0.5% weight/volume, yeast extract in an amount of about 0.3% to about 0.7% weight/volume, and glucose in an amount of about 1.0% to about 5% weight/volume.

40.-43. (canceled)

44. The method of claim 1, wherein the first population of microorganisms are present in the pre-fermentation medium at a concentration of about 5% to about 15% by volume.

45. The method of claim 1, wherein the first population of microorganisms comprise Rhizopus sp., Aspergillus niger, or both.

46. The method of claim 1, wherein incubating the inoculated pre-fermentation medium comprises incubating at a temperature of about 20° C. to about 40° C., for about 3 days to about 20 days.

47.-48. (canceled)

49. The method of claim 1, wherein incubating the inoculated pre-fermentation medium comprises incubating in an incubator capable of moving the pre-fermentation medium, wherein the incubator capable of moving the pre-fermentation medium is a shaking incubator, a rotating incubator, an oscillating incubator or a rocking incubator.

50.-51. (canceled)

52. The claim of claim 49, wherein the rotating incubator has a rotation speed of about 160 rpm to about 250 rpm.

53.-58. (canceled)

59. The method of claim 1, wherein the culture medium comprises peptone in an amount of about 0.1% to about 0.5% weight/volume, yeast extract in an amount of about 0.3% to about 0.7% weight/volume, and glucose in an amount of about 1% to about 5% weight/volume.

60.-63. (canceled)

64. The method of claim 1, wherein the second population of microorganisms are present in the culture medium at a concentration of about 5% to about 15% by volume, wherein the second population of microorganisms comprise Gluconacetobacter xylinus, Gluconacetobacter hansenii, Gluconobacter oxydans, Rhizobium sp., Sarcina sp., Pseudomonas sp., Achromobacter sp., Alcaligenes sp., Aerobacter sp., Azotobacter sp., Agrobacterium sp., Seudomonas cepacia, Campylobacter jejuni, or a combination thereof.

65. (canceled)

66. The method of claim 1, wherein incubating the inoculated culture medium comprises incubating at a temperature of about 20° C. to about 40° C., for about 3 days to about 20 days.

67.-68. (canceled)

69. The method of claim 1, wherein incubating the inoculated culture medium comprises incubating in an incubator capable of moving the inoculated culture medium, wherein the incubator capable of moving the inoculated culture medium is a shaking incubator, a rotating incubator, an oscillating incubator or a rocking incubator.

70.-73. (canceled)

74. The method of claim 1, further comprising harvesting the bacterial nanocellulose from the culture medium.

75. The method of claim 74, further comprising contacting the bacterial nanocellulose that is harvested with a base under conditions to remove residual first population of microorganisms, residual second population of microorganisms, residual culture medium, or a combination thereof, wherein contacting the bacterial nanocellulose that is harvested with the base comprises heating at about 70° C. to about 120° C. for about 90 minutes to about 150 minutes, and wherein the base is in aqueous form having a concentration of about 0.5% to about 8% by weight.

76.-79. (canceled)

80. A method of producing bacterial nanocellulose, the method comprising:
treating at least one cassava by-product to form particles of cassava byproduct having an average particle size of about 250 μm to about 420 μm;
preparing a pre-fermentation medium comprising the particles of cassava by-product;
inoculating the pre-fermentation medium with a first population of microorganism to produce an inoculated pre-fermentation medium, wherein the first population of microorganisms comprise Rhizopus sp., Aspergillus niger, or both;
incubating the inoculated pre-fermentation medium to produce an enriched supernatant;
collecting the enriched supernatant;
preparing a culture medium comprising the enriched supernatant;
inoculating the culture medium with a second population of microorganisms to produce an inoculated culture medium; and
incubating the inoculated culture medium to produce bacterial nanocellulose.

81.-85. (canceled)

86. The method of claim 80, wherein the pre-fermentation medium comprises peptone in an amount of about 0.1% to about 0.5% weight/volume, yeast extract in an amount of about 0.3% to about 0.7% weight/volume, and glucose in an amount of about 1.0% to about 5% weight/volume.

87.-89. (canceled)

90. The method of claim 80, wherein the first population of microorganisms are present in the pre-fermentation medium at a concentration about 5% to about 15% by volume.

91. (canceled)

92. The method of claim 80, wherein incubating the inoculated pre-fermentation medium comprises incubating at a temperature of about 20° C. to about 40° C., for about 3 days to about 20 days.

93.-100. (canceled)

101. The method of claim 80, wherein preparing the culture medium comprises adding at least one nitrogen source, at least one trace element, or both to the enriched supernatant, wherein the at least one nitrogen source is present in the culture medium at a concentration of about 0.1% to about 3% weight/volume, wherein the nitrogen source comprises organic nitrogen, and wherein the organic nitrogen source is peptone, yeast extract, tryptone, or a combination thereof.

102.-104. (canceled)

105. The method of claim 101, wherein the culture medium comprises peptone in an amount of about 0.1% to about 0.5%, yeast extract in an amount of about 0.3% to about 0.7%, and glucose in an amount of about 1% to about 5% weight/volume.

106.-108. (canceled)

109. The method of claim 80, wherein the second population of microorganisms are present in the culture medium at a concentration about 5% to about 15% by volume, and wherein the second population of microorganisms comprise Gluconacetobacter xylinus, Gluconacetobacter hansenii, Gluconobacter oxydans, Rhizobium sp., Sarcina sp., Pseudomonas sp., Achromobacter sp., Alcaligenes sp., Aerobacter sp., Azotobacter sp., Agrobacterium sp., Seudomonas cepacia, Campylobacter jejuni, or a combination thereof.

110. (canceled)

111. The method of claim 80, wherein incubating the inoculated culture medium comprises incubating at a temperature of about 20° C. to about 40° C., for about 3 days to about 20 days.

112.-115. (canceled)

116. The method of claim 80, wherein the incubator capable of moving the inoculated culture medium is a rotating incubator having a rotation speed of about 100 rpm to about 500 rpm.

117.-118. (canceled)

119. The method of claim 80, further comprising harvesting the bacterial nanocellulose from the culture medium.

120. The method of claim 119, further comprising contacting the bacterial nanocellulose that is harvested with a base under conditions to remove residual first population of microorganisms, residual second population of microorganisms, residual culture medium, or a combination thereof, wherein contacting the bacterial nanocellulose that is harvested with the base comprises heating at about 70° C. to about 120° C. for about 90 minutes to about 150 minutes, and wherein the base is in aqueous form having a concentration of about 0.5% to about 8% by weight.

121.-124. (canceled)

125. A method of making a culture medium, the method comprising:
contacting cassava bagasse with a catalyst to form a reaction mixture, wherein the catalyst is an acid catalyst, an enzymatic catalyst, or a combination thereof;
subjecting the reaction mixture to conditions sufficient to hydrolyze at least a portion of the cassava bagasse to produce cassava bagasse hydrolysate;
preparing a pre-fermentation medium comprising the cassava bagasse hydrolysate;
inoculating the pre-fermentation medium with a population of microorganisms to produce an inoculated pre-fermentation medium, wherein the population of microorganisms comprise Rhizopus sp., Aspergillus niger, or both;
incubating the inoculated pre-fermentation medium to produce an enriched supernatant;
collecting the enriched supernatant; and
preparing the culture medium with the enriched supernatant.

126. The method of claim 125, further comprising fragmenting the cassava bagasse before contacting with the catalyst, wherein the cassava bagasse has an average particle size of about 250 μm to about 420 μm after the fragmenting.

127.-129. (canceled)

130. The method of claim 125, wherein preparing the pre-fermentation medium comprises adding at least one nitrogen source to the cassava bagasse hydrolysate, wherein the at least one nitrogen source is present in the pre-fermentation medium in an amount from about 0.1% to about 3%, weight/volume.

131. (canceled)

132. The method of claim 125, wherein the pre-fermentation medium comprises peptone in an amount of about 0.1% to about 0.5% weight/volume, yeast extract in an amount of about 0.3% to about 0.7% weight/volume, and glucose in an amount of about 1.0% to about 5%.

133. (canceled)

134. The method of claim 125, wherein the population of microorganisms are present in the pre-fermentation medium at a concentration from about 5% to about 15% by volume.

135. The method of claim 125, wherein incubating the inoculated pre-fermentation medium comprises incubating at a temperature of about 20° C. to about 40° C.

136. (canceled)

137. The method of claim 125, wherein preparing the culture medium comprises adding at least one nitrogen source, at least one trace element, or both to the enriched supernatant.

138.-147. (canceled)
